(19) United States
(12) Patent Application Publication
(10) Pub. No.: US 2026/0034314 A1
(43) Pub. Date: Feb. 5, 2026

(54) Composite Safety Self-Destructing Injection Syringe

(71) Applicant: Guangdong Hongshan Medical Device Technology Co., Ltd., Huizhou (CN)

(72) Inventors: Xurong Huang, Huizhou (CN); Haichuan Huang, Huizhou (CN)

(21) Appl. No.: 18/789,138

(22) Filed: Jul. 30, 2024

(51) Int. Cl. A61M 5/50

(52) U.S. Cl. CPC A61M 5/508

(57) ABSTRACT

A composite safety self-destructing injection syringe comprises an injection tube, a needle seat, a sealing rubber plug, and an injection rod; an inside of the injection tube is provided with a sealing ring, and a rear end of the needle seat contacts and limits the position with the sealing ring by a through hole; a spring sleeve is connected between the sealing rubber plug and the injection rod, and outer side walls at both ends of the spring sleeve are respectively clamped with the sealing rubber plug and the injection rod; an end surface of the spring sleeve close to the sealing rubber plug is provided with a clamping joint, and an end of the injection rod away from the spring sleeve is movably connected with a rear cover. The self-destructing function will not be triggered when the injection rod is pushed to the zero position.

### TECHNICAL FIELD

The invention belongs to the technical field of syringes, and particularly relates to a composite safety self-destructing injection syringe.

### BACKGROUND ART

With the widespread use of injection syringes, in order to avoid cross infection between medical patients, more and more self-destructing injection syringes appear on the market. Self-destructing injection syringes are disposable injection syringe products that can prevent reuse after use. There are various types. After the injection is completed, manual or mechanical force is used to prevent the push rod from being withdrawn from the injection syringe, achieving self-locking, or the sealing performance of the piston is destroyed, or the push rod of the syringe can be pushed out, ultimately achieving the purpose of preventing reuse.

Currently, there are many retractable self-destructing injection syringes on the market, which allow the needle to retract into the injection syringe after the injection is completed to achieve a self-destructing function. However, the safety performance of this type of retractable self-destructing injection syringe restricts the widespread promotion of this product.

When using an injection syringe, users usually need to draw out medicine multiple times for dispensing. Because the air in the injection syringe needs to be completely discharged, the injection rod must be pushed to the zero position of the injection syringe. This operation is prone to safety problems. When the injection rod is pushed to the zero position, the self-destructing function of the injection syringe is triggered, rendering the injection syringe unusable. On the other hand, when users use this type of injection syringe to inject medicine into patients, it is also easy to make operating errors, triggering the self-destructing function of the injection syringe during the injection process, causing the needle to be accidentally pulled out directly from the patient's body, which may cause medical accidents.

### SUMMARY OF THE INVENTION

The technical problem to be solved by the invention is to change the position structure of the self-destructing function so that the self-destructing function will not be triggered when the injection rod is pushed to the zero position, and the injection rod can be repeatedly pushed to the zero position for use, thereby reducing the residual liquid medicine inside the injection tube.

In order to solve the above technical problems, the technical solution provided by the invention is as follows: a composite safety self-destructing injection syringe, comprising an injection tube, a needle seat, a sealing rubber plug, and an injection rod; the needle seat is slidably provided in the injection tube and movably connected to an injection end of the injection tube along a length direction of the injection tube; the sealing rubber plug is slidably provided in the injection tube by the pushing of the injection rod, and the sealing rubber plug is movably connected to a feed end of the injection rod; wherein an inside of the injection tube is provided with a sealing ring, and a rear end of the needle seat contacts and limits the position with the sealing ring by a through hole; a spring sleeve is connected between the sealing rubber plug and the injection rod, and outer side walls at both ends of the spring sleeve are respectively clamped with the sealing rubber plug and the injection rod; an end surface of the spring sleeve close to the sealing rubber plug is provided with a clamping joint, and an end of the injection rod away from the spring sleeve is movably connected with a rear cover.

Further, an inner side wall of an end of the injection tube close to the needle seat is provided with a fixing groove; both sides of the fixing groove are fixed with first convex ribs, and the first convex ribs are symmetrically arranged up and down with the fixing groove as a center; an outer side wall of the sealing ring is provided with a second convex rib, and the second convex rib is clamped in the fixing groove; the through hole is arranged on an end surface of the sealing ring and is expanded in an inclined manner toward an end close to the needle seat; a middle of the through hole is provided with a buffer cavity.

Further, an outer side wall of an end of the spring sleeve away from the clamping joint is fixed with a first clamping block; an inner side wall of an end of the injection rod close to the spring sleeve is fixed with second clamping blocks, and the second clamping blocks are arranged in a ring shape with uniform intervals; the clamping joint is arranged in a tapered diamond shape.

Further, a side wall of the buffer cavity and a side wall of the through hole are arranged to be inclined in an opposite direction, and an end surface of the sealing ring away from the needle seat is arranged to be an inclined surface with a concave center.

Further, a middle of the inner side wall of the sealing rubber plug is provided with a clamping groove, and the sealing rubber plug is connected to the feed end of the injection rod through the clamping groove; an inner side wall of an end of the sealing rubber plug close to the clamping joint is provided with a widening groove, and the widening groove is arranged adjacent to the clamping groove; an outer side wall of the sealing rubber plug is provided with a fourth convex rib.

Further, an outer side wall of the spring sleeve is wound with a support spring, and the support spring is limited and compressed between an inner end surface of the feed end of the injection rod and the first clamping block by the second clamping blocks.

Further, a bottom end of the needle seat is provided with a stuck groove; an outer side wall of an end of the needle seat close to the stuck groove is arranged to be an outwardly convex inclined surface, and is matched with the inclined surface of an inner side wall of the through hole; an end surface of the outwardly convex inclined surface of the needle seat forms a first step.

Further, an inner side wall of the rear cover is provided with a third convex rib, and the third convex rib is clamped with the outer side wall of the spring sleeve through the second clamping blocks.

Further, the injection rod is arranged in a cavity, and a side wall of an end of the injection rod close to the rear cover is provided with a vent hole.

After adopting the above structure, the invention has the following beneficial effects in view of that the self-destructing function is accidentally triggered when the liquid medicine in the injection syringe is repeatedly pushed to the zero position;
(1) limiting the sealing ring: first convex ribs are added on both sides of the sealing ring to improve the limiting effect of the sealing ring and increase the limiting effect on the needle seat, which supports the sealing rubber plug to fit the sealing ring and realize repeated pushing of the liquid medicine;
(2) when the injection rod is pushed forward, the widening groove inside the sealing rubber plug increases the elastic space, making it easier for the sealing rubber plug to push the liquid medicine to the injection zero position, reducing the residual amount of liquid medicine in the injection tube; at the same time, the sealing ring and the needle seat are in contact with each other through the inclined surface, which plays a role of negative pressure to prevent liquid leakage, and positive pressure is generated in the injection tube to prevent liquid leakage;
(3) the diamond-shaped taper clamping joint is inserted into the stuck groove of the needle seat to activate the self-destructing function; when the self-destructing function is activated, the friction force of the stuck connector locking into the stuck groove is reduced, and when it is pulled back, the friction locking force between the stuck connector and the stuck groove is increased, making it difficult to fall off;
(4) by adding a third convex rib on the inner wall of the rear cover, after the self-destructing function is activated, the needle seat retracts to the third convex rib of the rear cover, and the first clamping block slides and engages through the third convex rib, and the retracted needle is positioned in the injection rod, so that the needle is not exposed and secondary injury is avoided.

### BRIEF DESCRIPTION OF ACCOMPANY DRAWINGS

The accompanying drawings are used to provide a further understanding of the invention and constitute a part of the specification. They are used together with the embodiments of the invention to explain the invention and do not constitute a limitation to the invention.

FIG. 1 is a half-section view of the composite safety self-destructing injection syringe according to the invention;

FIG. 2 is a schematic diagram of the structure of the injection tube of the composite safety self-destructing injection syringe according to the invention;

FIG. 3 is an enlarged view of part A in FIG. 2;

FIG. 4 is a schematic diagram of the structure of the spring sleeve of the composite safety self-destructing injection syringe according to the invention;

FIG. 5 is an enlarged view of part A in FIG. 4;

FIG. 6 is a half-section view of the sealing ring of the composite safety self-destructing injection syringe according to the invention;

FIG. 7 is a half-section view of the needle seat of the composite safety self-destructing injection syringe according to the invention;

FIG. 8 is a half-section view of the sealing rubber plug of the composite safety self-destructing injection syringe according to the invention;

FIG. 9 is a schematic diagram of the connection between the needle seat and the sealing ring of the composite safety self-destructing injection syringe according to the invention;

FIG. 10 is a schematic diagram of the initial state of self-destruction of the composite safety self-destructing injection syringe according to the invention;

FIG. 11 is a diagram showing a contracted state of the needle seat of the composite safety self-destructing injection syringe according to the invention after self-destruction is completed;

FIG. 12 is an enlarged view of part C in FIG. 11.

In the drawings: 1 refers to the injection tube; 2 refers to the needle seat; 3 refers to the sealing tuber plug; 4 refers to the injection rod; 5 refers to the sealing ring; 6 refers to the spring sleeve; 7 refers to the through hole; 8 refers to the clamping joint; 9 refers to the rear cover; 10 refers to the fixing groove; 11 refers to the first convex rib; 12 refers to the second convex rib; 13 refers to the buffer cavity; 14 refers to the first clamping block; 15 refers to the second clamping block; 16 refers to the clamping groove; 17 refers to the widening groove; 18 refers to the fourth convex rib; 19 refers to the support spring; 20 refers to the stuck groove; 21 refers to the first step; 22 refers to the third convex rib; 23 refers to the vent hole.

### SPECIFIC EMBODIMENT OF THE INVENTION

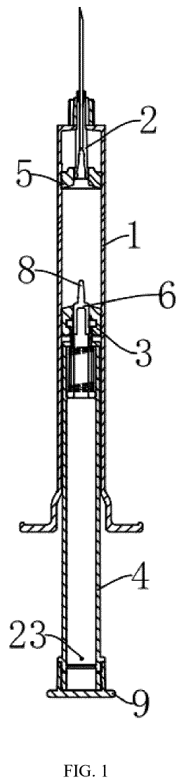

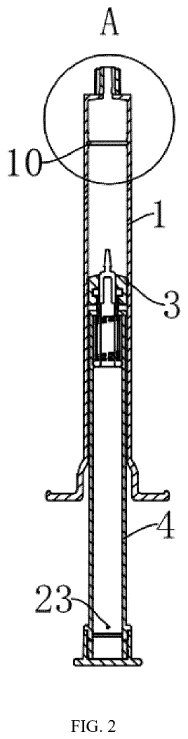

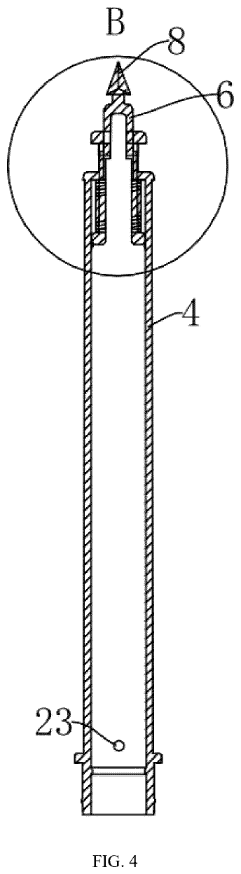

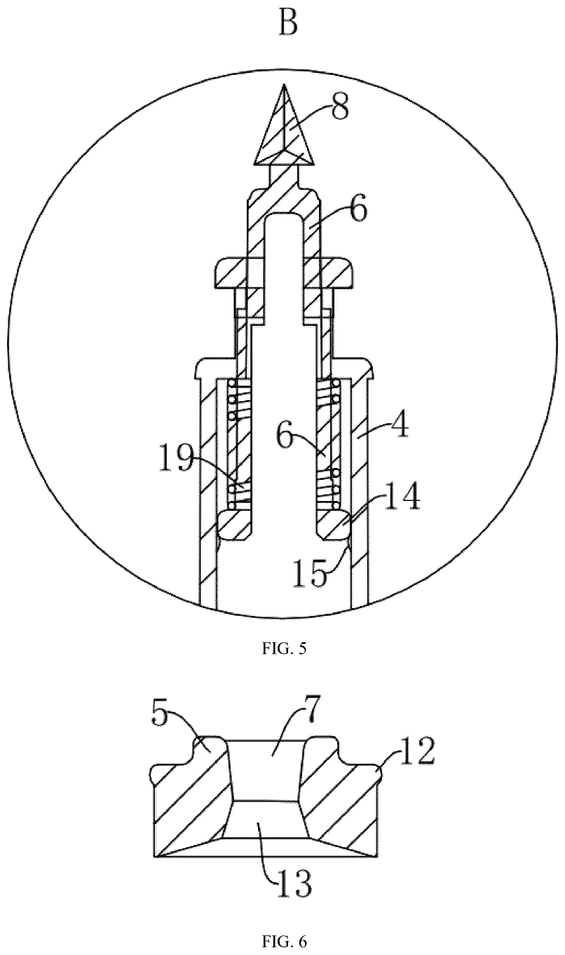

As shown in FIGS. 1, 2, 4, 5, and 10, a composite safety self-destructing injection syringe, comprising an injection tube 1, a needle seat 2, a sealing rubber plug 3, and an injection rod 4; the needle seat 2 is slidably provided in the injection tube 1 and movably connected to an injection end of the injection tube 1 along a length direction of the injection tube 1; the sealing rubber plug 3 is slidably provided in the injection tube 1 by the pushing of the injection rod 4, and the sealing rubber plug 3 is movably connected to a feed end of the injection rod 4; wherein an inside of the injection tube 1 is provided with a sealing ring 5, and a rear end of the needle seat 2 contacts and limits the position with the sealing ring 5 by a through hole 7; a spring sleeve 6 is connected between the sealing rubber plug 3 and the injection rod 4, and outer side walls at both ends of the spring sleeve 6 are respectively clamped with the sealing rubber plug 3 and the injection rod 4; an end surface of the spring sleeve 6 close to the sealing rubber plug 3 is provided with a clamping joint 8, and an end of the injection rod 4 away from the spring sleeve 6 is movably connected with a rear cover 9. When the injection rod 4 drives the sealing rubber plug 3 to repeatedly inject liquid medicine into the injection tube 1, the sealing ring 5 contacts the end surface of the sealing rubber plug 3, pushes the liquid medicine to the zero position, and limits the position of the spring sleeve 6 at the same time, so that the self-destructing function will not be triggered.

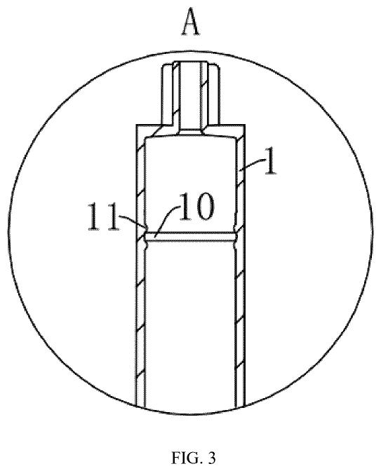

As shown in FIGS. 1, 3, and 6, in order to achieve repeated injection and increase the positioning effect of the sealing ring 5 in the injection tube 1, an inner side wall of an end of the injection tube 1 close to the needle seat 2 is provided with a fixing groove 10; both sides of the fixing groove 10 are fixed with first convex ribs 11, and the first convex ribs 11 are symmetrically arranged up and down with the fixing groove 10 as a center; an outer side wall of the sealing ring 5 is provided with a second convex rib 12, and the second convex rib 12 is clamped in the fixing groove 10. At the same time, the first convex ribs 11 on both sides limit the second convex rib 12, so that the sealing ring 5 is closely matched with the inner wall of the injection tube 1 without displacement.

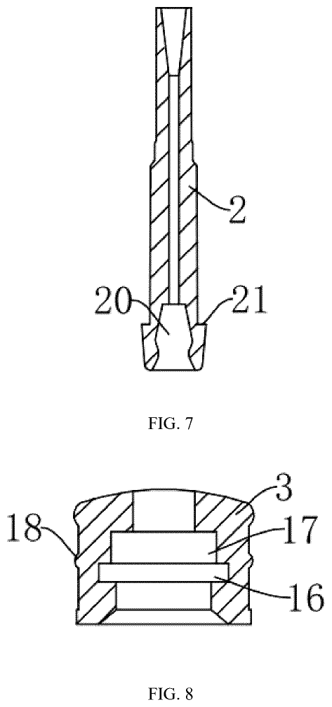

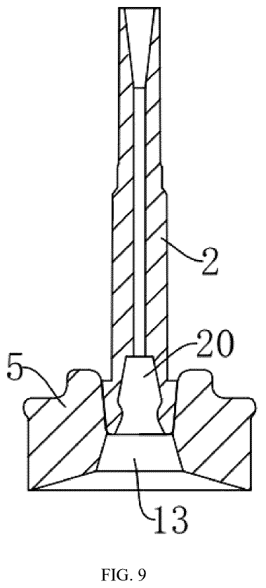

As shown in FIGS. 1 and 6-9, in order to increase the fit between the needle seat 2 and the sealing ring 5 and avoid that the connection between the needle seat 2 and the sealing ring 5 leaks during injection, the through hole 7 is arranged on an end surface of the sealing ring 5 and is expanded in an inclined manner toward an end close to the needle seat 2. An outer side wall of an end of the needle seat 2 close to the stuck groove 20 is arranged to be an outwardly convex inclined surface, and is matched with the inclined surface of an inner side wall of the through hole 7; an end surface of the outwardly convex inclined surface of the needle seat 2 forms a first step 21. The side wall of the through hole 7 has an inclination, which is cooperated with the inclination of the bottom of the needle seat 2 to play a role of negative pressure and no leakage. When the injection rod 4 is pushed, positive pressure is generated in the tube and no leakage is generated.

In order to reduce the residual amount of liquid medicine in the injection tube 1 during injection, a middle of the inner side wall of the sealing rubber plug 3 is provided with a clamping groove 16, and the sealing rubber plug 3 is connected to the feed end of the injection rod 4 through the clamping groove 16; an inner side wall of an end of the sealing rubber plug 3 close to the clamping joint 8 is provided with a widening groove 17, and the widening groove 17 is arranged adjacent to the clamping groove 16; an outer side wall of the sealing rubber plug 3 is provided with a fourth convex rib 18. By widening the internal aperture of the sealing rubber plug 3 and increasing the elastic space of the sealing rubber plug 3, the sealing rubber plug 3 can be pushed to the injection zero position more easily.

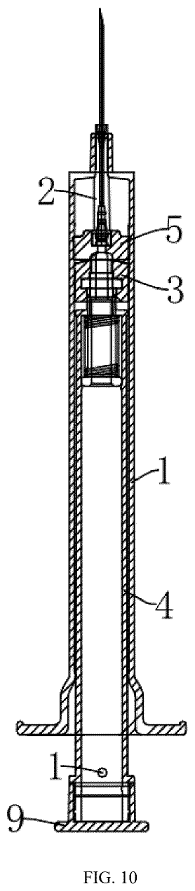

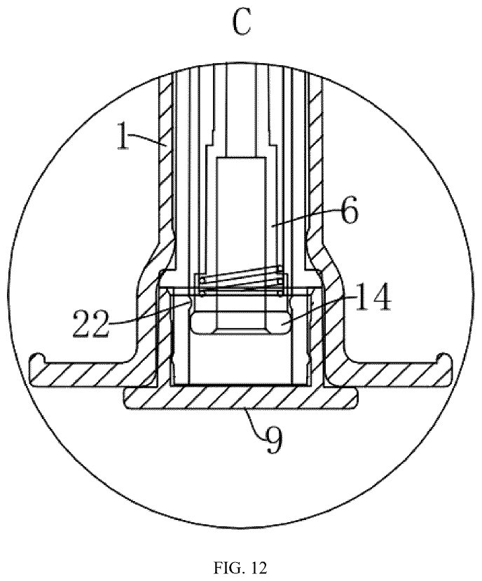

As shown in FIGS. 10-12, in order to control the self-destructing function, an outer side wall of an end of the spring sleeve 6 away from the clamping joint 8 is fixed with a first clamping block 14; an inner side wall of an end of the injection rod 4 close to the spring sleeve 6 is fixed with second clamping blocks 15, and the second clamping blocks 15 are arranged in a ring shape with uniform intervals; an outer side wall of the spring sleeve 6 is wound with a support spring 19; a middle of the through hole 7 is provided with a buffer cavity 13; a side wall of the buffer cavity 13 and a side wall of the through hole 7 are arranged to be inclined in an opposite direction, and an end surface of the sealing ring 5 away from the needle seat 2 is arranged to be an inclined surface with a concave center; the clamping joint 8 is arranged in a tapered diamond shape; the support spring 19 is limited and compressed between an inner end surface of the feed end of the injection rod 4 and the first clamping block 14 by the second clamping blocks 15. In the state of repeated injection, the first clamping block 14 at the bottom of the spring sleeve 6 is limited at the front end of the injection rod 4 by the second clamping blocks 15, and at the same time, the support spring 19 is compressed and limited. When the sealing ring 5 is fitted with the sealing rubber plug 3, the clamping joint 8 is in the buffer cavity 13 and does not contact the needle seat 2. When the self-destructing function is activated, the injection rod 4 is pushed forward, and the clamping joint 8 is inserted into the stuck groove 20 at the bottom of the needle seat 2 to realize the connection between the spring sleeve 6 and the needle seat 2. The injection rod 4 is continuously pushed forward to make the spring sleeve 6 disengage from the clamping of the second clamping blocks 15, and the sealing ring 5 and the sealing rubber plug 3 are fixed to the inner wall of the injection end of the injection tube 1. The elastic force of the support spring 19 and the air pressure are used to push the spring sleeve 6 into the cavity of the injection rod 4 to realize the retraction of the needle seat 2.

In order to fix the retracted needle seat 2, an inner side wall of the rear cover 9 is provided with a third convex rib 22, and the third convex rib 22 is clamped with the outer side wall of the spring sleeve 6 through the second clamping blocks 15. The injection rod 4 is arranged in a cavity, and a side wall of an end of the injection rod 4 close to the rear cover 9 is provided with a vent hole 23. The support spring 19 pushes the spring sleeve 6 toward one end of the rear cover 9 in the cavity of the injection rod 4. The first clamping block 14 of the spring sleeve 6 is slidably clamped with the third convex rib 22 to fix the needle seat 2 to ensure that the needle will not fall back and the injection rod 4 will not be exposed. The vent hole 23 facilitates the needle to rebound.

When the operator needs to use the injection syringe to dispense medicine, he needs to push the injection rod 4 to the zero position, and the sealing ring 5 contacts the end surface of the sealing rubber plug 3. At this time, the clamping joint 8 of the spring sleeve 6 is in the buffer cavity 13 and will not be clamped with the stuck groove 20 on the needle seat 2, so as to avoid triggering the self-destructing function and achieve safe dispensing of medicine.

When the medicine is prepared, it is necessary to inject the medicine into the patient's body. As shown in FIG. 10, the injection rod 4 is pushed to the start state, and the clamping joint 8 of the spring sleeve 6 is in the buffer cavity 13 and will not be clamped with the stuck groove 20 on the needle seat 2, and the self-destructing function will not be triggered, thereby ensuring the safety of the injection and avoiding medical accidents.

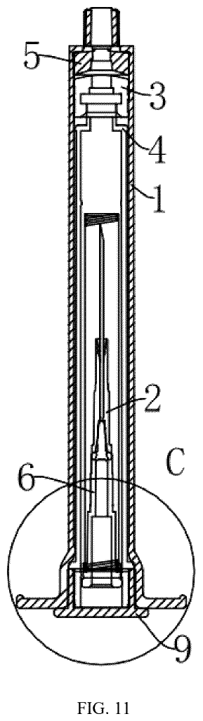

After the injection is completed and the injection syringe is pulled out of the human body, the injection syringe needs to be self-destructed. As shown in FIG. 11, the injection rod 4 is pushed toward the injection end of the injection tube 1. The end of the injection rod 4 squeezes the sealing ring 5, so that the second ribs 12 on both sides of the sealing ring 5 are separated from the clamping of the fixing groove 10. As the injection rod 4 moves toward the injection end of the injection tube 1, the compressed air generated by the gap between the sealing ring 5 and the injection tube 1 pushes the first step surface, and the upward elastic stress of the needle seat 2 becomes larger and larger until the clamping joint 8 passes through the buffer cavity 13 and is clamped in the stuck groove 20. The injection rod 4 is sequentially pushed downward to separate the end of the spring sleeve 6 from the clamping of the second clamping blocks 15. The sealing ring 5 and the sealing rubber plug 3 are respectively limited at the injection end of the injection tube 1 by the second ribs 12 and the fourth rib 18 on the outer wall, and will not return.

Under the support of the support spring 19, as shown in FIG. 11, the elastic stress of the spring sleeve 6 plus the elastic stress of the compressed air on the needle seat 2 causes the spring sleeve 6 and the needle seat 2 to be ejected into the cavity of the injection rod 4 together. After the support spring 19 is stretched, it can press the top of the spring sleeve 6 in the cavity of the injection rod 4, thereby ensuring that the needle will not fall back and the injection rod 4 will not be exposed. Since the injection rod 4 is provided with a vent hole 23, it is more convenient for the needle to rebound.

Through the composite injection syringe assembly structure of the invention, the operator can have a clear stage division when operating the injection syringe to implement injection and self-destructing, and each stage has clear operation feedback, which can ensure the safety of injection implementation and achieve the reliability of self-destructing.

Although the embodiments of the invention have been shown and described, for those of ordinary skill in the art, it can be understood that various changes, modifications, and substitutions can be made to these embodiments without departing from the principle and spirit of the invention. The protection scope of the invention is defined by the appended claims and the equivalents thereof. All in all, structural methods and embodiments similar to the technical solution without deviating from the purpose of the invention made by those of ordinary skill in the art without creative design shall all fall within the protection scope of the invention.

1. A composite safety self-destructing injection syringe, comprising an injection tube, a needle seat, a sealing rubber plug, and an injection rod; the needle seat is slidably provided in the injection tube and movably connected to an injection end of the injection tube along a length direction of the injection tube; the sealing rubber plug is slidably provided in the injection tube by the pushing of the injection rod, and the sealing rubber plug is movably connected to a feed end of the injection rod; wherein an inside of the injection tube is provided with a sealing ring, and a rear end of the needle seat contacts and limits the position with the sealing ring by a through hole; a spring sleeve is connected between the sealing rubber plug and the injection rod, and outer side walls at both ends of the spring sleeve are respectively clamped with the sealing rubber plug and the injection rod; an end surface of the spring sleeve close to the sealing rubber plug is provided with a clamping joint, and an end of the injection rod away from the spring sleeve is movably connected with a rear cover.

2. The composite safety self-destructing injection syringe of claim 1, wherein an inner side wall of an end of the injection tube close to the needle seat is provided with a fixing groove; both sides of the fixing groove are fixed with first convex ribs, and the first convex ribs are symmetrically arranged up and down with the fixing groove as a center; an outer side wall of the sealing ring is provided with a second convex rib, and the second convex rib is clamped in the fixing groove; the through hole is arranged on an end surface of the sealing ring and is expanded in an inclined manner toward an end close to the needle seat; a middle of the through hole is provided with a buffer cavity.

3. The composite safety self-destructing injection syringe of claim 1, wherein an outer side wall of an end of the spring sleeve away from the clamping joint is fixed with a first clamping block; an inner side wall of an end of the injection rod close to the spring sleeve is fixed with second clamping blocks, and the second clamping blocks are arranged in a ring shape with uniform intervals; the clamping joint is arranged in a tapered diamond shape.

4. The composite safety self-destructing injection syringe of claim 1, wherein a side wall of the buffer cavity and a side wall of the through hole are arranged to be inclined in an opposite direction, and an end surface of the sealing ring away from the needle seat is arranged to be an inclined surface with a concave center.

5. The composite safety self-destructing injection syringe of claim 1, wherein a middle of the inner side wall of the sealing rubber plug is provided with a clamping groove, and the sealing rubber plug is connected to the feed end of the injection rod through the clamping groove; an inner side wall of an end of the sealing rubber plug close to the clamping joint is provided with a widening groove, and the widening groove is arranged adjacent to the clamping groove; an outer side wall of the sealing rubber plug is provided with a fourth convex rib.

6. The composite safety self-destructing injection syringe of claim 1, wherein an outer side wall of the spring sleeve is wound with a support spring, and the support spring is limited and compressed between an inner end surface of the feed end of the injection rod and the first clamping block by the second clamping blocks.

7. The composite safety self-destructing injection syringe of claim 1, wherein a bottom end of the needle seat is provided with a stuck groove; an outer side wall of an end of the needle seat close to the stuck groove is arranged to be an outwardly convex inclined surface, and is matched with the inclined surface of an inner side wall of the through hole; an end surface of the outwardly convex inclined surface of the needle seat forms a first step.

8. The composite safety self-destructing injection syringe of claim 1, wherein an inner side wall of the rear cover is provided with a third convex rib, and the third convex rib is clamped with the outer side wall of the spring sleeve through the second clamping blocks.

9. The composite safety self-destructing injection syringe of claim 1, wherein the injection rod is arranged in a cavity, and a side wall of an end of the injection rod close to the rear cover is provided with a vent hole.
